(19) United States
(12) Patent Application Publication
(10) Pub. No.: US 2014/0308275 A1
(43) Pub. Date: Oct. 16, 2014

(54) METHODS FOR DIAGNOSING AND TREATING MYHRE SYNDROME

(71) Applicants: Valérie Cormier-Daire, Paris (FR); Carine Le Goff, Paris (FR); Arnold Munnich, Paris (FR)

(72) Inventors: Valérie Cormier-Daire, Paris (FR); Carine Le Goff, Paris (FR); Arnold Munnich, Paris (FR)

(73) Assignees: INSERM (INSTITUT NATIONAL DE LA SANTE ET DE LA RECHERCHE MEDICALE, Paris (FR); UNIVERSITE PARIS DESCARTES - PARIS V, Paris (FR)

(21) Appl. No.: 14/235,123

(22) PCT Filed: Jul. 27, 2012
(86) PCT No.: PCT/EP2012/064763

§ 371 Date: Apr. 8, 2014

(30) Foreign Application Priority Data
Jul. 27, 2011 (EP) 11305978.6

(51) Int. Cl. C12Q 1/68; A61K 31/713; G01N 33/68; A61K 38/17; A61K 39/395

(52) U.S. Cl. CPC C12Q 1/6883; A61K 38/1709; A61K 39/3955; G01N 33/6872; A61K 31/713
USPC 424/133.1; 514/8.9; 424/158.1; 435/6.11; 435/7.1; 514/44 A; 506/2

(57) ABSTRACT

The present invention relates to methods for diagnosing and treating Myhre Syndrome. The invention provides a method for diagnosing or predicting Myhre Syndrome, or a risk of Myhre Syndrome, in a subject, which method comprises detecting a mutation in SMAD4 gene, as compared to a control population, wherein the presence of said mutation is indicative of Myhre Syndrome or of a risk of Myhre Syndrome. The present invention also relates to an inhibitor of the SMAD4-mediated TGFβ/BMP signalling pathway for use in the treatment of Myhre Syndrome.

### FIELD OF THE INVENTION

The present invention relates to methods for diagnosing and treating Myhre Syndrome.

### BACKGROUND OF THE INVENTION

Myhre Syndrome (MS) is a well defined entity characterized by pre and postnatal short stature, brachydactyly, facial dysmorphism, (short palpebral fissures, maxillary hypoplasia, prognathism, short philtrum), thick skin, generalized muscle hypertrophy, and restricted joint mobility. Deafness of mixed conductive and sensory type is consistently observed in older patients. Other features include developmental delay with mental retardation or/and behavioral disturbance, cardiac defects, cryptorchidism and bone anomalies. Skeletal manifestations include thickened calvarium, cone-shaped epiphyses, shortened tubular bones, hypoplastic iliac wings, broad ribs and large and flattened vertebrae with large pedicles. Differential diagnoses include acromicric and geleophysic dysplasias (AD/GD), also characterized by short stature, brachydactyly, joint limitation and skin thickening. MS can be distinguished from AD and GD by facial features, the presence of developmental delay, deafness and distinct skeleton features. We have recently identified ADAMTSL2 mutations in GD and demonstrated a direct involvement of ADAMTSL2 in TGFβ bioavailability. The molecular basis of AD are still unknown. All reported MS cases are sporadic and advanced paternal age at the time of fecondation has been reported in a few instances supporting de novo dominant mutations.

### SUMMARY OF THE INVENTION

The invention provides a method for diagnosing or predicting Myhre Syndrome, or a risk of Myhre Syndrome, in a subject, which method comprises detecting a mutation in SMAD4 gene, as compared to a control population, wherein the presence of said mutation is indicative of Myhre Syndrome or of a risk of Myhre Syndrome.

The present invention also relates to the present invention relates to an inhibitor of the SMAD4-mediated TGFβ/BMP signalling pathway for use in the treatment of Myhre Syndrome

### DETAILED DESCRIPTION OF THE INVENTION

Myhre syndrome (OMIM #139210, MS) is a developmental disorder characterized by short stature, short hands and feet, facial dysmorphism, muscular hypertrophy, deafness and cognitive delay. Using exome sequencing in MS cases, the inventors selected mothers against DPP homolog 4 (SMAD4) as a candidate gene based on its pivotal role in BMP and TGFβ signalling. They identified 3 distinct heterozygous missense SMAD4 mutations at codon Isoleucine 500 in 11 MS cases. All 3 mutations were located in the Mad Homology 2 (MH2) domain. They found increased levels of SMAD4 and enhanced SMAD2/3 and SMAD1/5/8 phosphorylation in patient fibroblasts. These results provide evidence of dysregulation of BMP and TGFβ signalling as the underlying mechanism of MS phenotype.

As used herein, the term “SMAD4” has its general meaning in the art. SMAD4 (also known as MADH4 and DPC4) represents the most unique member of the Smad family. This protein acts as a shared hetero-oligomerization partner in complexes with the pathway-restricted Smads (Lagna et al., Nature, 1996, 383,832-836; Zhang et al., Curr. Biol., 1997, 7, 270-276). Recently it has been demonstrated that although SMAD4 does not interact with the TGF-beta receptor, it does perform two distinct functions within the Smad signaling cascade. Through its N-terminus SMAD4 promotes the binding of the Smad complex to DNA, and through its C-terminus it provides an activation signal required for the Smad complex to stimulate transcription (Liu et al., Genes Dev., 1997, 11, 3157-3167). SMAD4 was first isolated as a possible tumor suppressor gene during studies of pancreatic carcinomas. Homo sapiens SMAD4 gene is localized to chromosome 18q21.1 (Hahn et al., Science, 1996, 271, 350-353), the sequence of which is deposited in Genebank under accession number NG_{—}013013.1. Typically a nucleic acid sequence encoding for SMAD4 is shown as SEQ ID NO:1. The corresponding amino acid sequence is deposited in GenPept database under accession number NP_{—}005350.1 (SEQ ID NO:2). The Mad Homology 2 (MH2) domain corresponds to the region ranging from position 317 to position 530 in the amino acid sequence of SMAD4 (SEQ ID NO:2).

Diagnostic Methods of the Invention:

The inventors have shown that mutations found in SMAD4 gene are associated with Myhre Syndrome.

Therefore, the invention provides a method for diagnosing or predicting Myhre Syndrome, or a risk of Myhre Syndrome, in a subject, which method comprises detecting a mutation in SMAD4 gene, as compared to a control population, wherein the presence of said mutation is indicative of Myhre Syndrome or of a risk of Myhre Syndrome.

Preferably, the SMAD4 mutation according to the invention is found in the Mad Homology 2 (MH2) domain. More preferably, the mutation of SMAD4 according to the invention is located at the codon encoded for the isoleucine at position 500 in the SMAD4 protein. Even more preferably the SMAD4 mutation according to the invention is selected in from the group consisting of c.1498 A>G, c.1499 T>C, and c. 1500 A>G in SEQ ID NO:1.

According to a first embodiment, said mutation may be detected by analyzing a SMAD4 nucleic acid molecule. In the context of the invention, SMAD4 nucleic acid molecules include mRNA, genomic DNA and cDNA derived from mRNA. DNA or RNA can be single stranded or double stranded. These may be utilized for detection by amplification and/or hybridization with a probe, for instance.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood, plasma, serum, lymph, etc. DNA may be extracted using any methods known in the art, such as described in Sambrook et al., 1989. RNA may also be isolated, for instance from tissue biopsy, using standard methods well known to the one skilled in the art such as guanidium thiocyanate-phenol-chloroform extraction.

SMAD4 mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular SMAD4 mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in SMAD4 sequence.

Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

Therefore, useful nucleic acid molecules, in particular oligonucleotide probes or primers, according to the present invention include those which specifically hybridize the regions where the mutations are located.

Oligonucleotide probes or primers may contain at least 10, 15, 20 or 30 nucleotides. Their length may be shorter than 400, 300, 200 or 100 nucleotides.

According to a second embodiment said mutation in the SMAD4 gene may be detected at the protein level.

Accordingly, a mutation of SMAD4 according to the invention is preferably selected from the group consisting of mutations which result in 1500M, 1500T and 1500V mutants of SMAD4 (SEQ ID NO:2).

Said mutation may be detected according to any appropriate method known in the art. In particular a sample, such as a tissue biopsy, obtained from a subject may be contacted with antibodies specific of the mutated form of SMAD4, i.e. antibodies that are capable of distinguishing between a mutated form of SMAD4 and the wild-type protein (or any other protein), to determine the presence or absence of a SMAD4 specified by the antibody.

Antibodies that specifically recognize a mutated SMAD4 also make part of the invention. The antibodies are specific of mutated SMAD4, that is to say they do not cross-react with the wild-type SMAD4.

The antibodies of the present invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering mutated SMAD4 subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988) which is hereby incorporated in the references.

A “monoclonal antibody” in its various grammatical forms refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified mutated SMAD4 into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Antibodies raised against mutated SMAD4 may be cross reactive with wild-type SMAD4. Accordingly a selection of antibodies specific for mutated SMAD4 is required. This may be achieved by depleting the pool of antibodies from those that are reactive with the wild-type SMAD4, for instance by submitting the raised antibodies to an affinity chromatography against wild-type SMAD4.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Probe, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probe, primers, aptamers or antibodies of the invention, is intended to encompass direct labelling of the the probe, primers, aptamers or antibodies of the invention by coupling (i.e., physically linking) a detectable substance to the the probe, primers, aptamers or antibodies of the invention, as well as indirect labeling of the probe, primers, aptamers or antibodies of the invention by reactivity with another reagent that is directly labeled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labeled secondary antibody and end-labeling of a DNA probe with biotin such that it can be detected with fluorescently labeled streptavidin. An antibody or aptamer of the invention may be labelled with a radioactive molecule by any method known in the art. For example radioactive molecules include but are not limited radioactive atom for scintigraphic studies such as I123, I124, In111, Re186, Re188.

Kits of the Invention:

According to another aspect of the invention, the SMAD4 mutation is detected by contacting the DNA of the subject with a nucleic acid probe, which is optionally labeled.

Primers may also be useful to amplify or sequence the portion of the SMAD4 gene containing the mutated positions of interest.

Such probes or primers are nucleic acids that are capable of specifically hybridizing with a portion of the SMAD4 gene sequence containing the mutated positions of interest. That means that they are sequences that hybridize with the portion mutated SMAD4 nucleic acid sequence to which they relate under conditions of high stringency.

The present invention further provides kits suitable for determining at least one of the mutations of the SMAD4 gene.

The kits may include the following components:

(i) a probe, usually made of DNA, and that may be pre-labelled. Alternatively, the probe may be unlabelled and the ingredients for labelling may be included in the kit in separate containers; and

(ii) hybridization reagents: the kit may also contain other suitably packaged reagents and materials needed for the particular hybridization protocol, including solid-phase matrices, if applicable, and standards.

In another embodiment, the kits may include:

(i) sequence determination or amplification primers: sequencing primers may be pre-labelled or may contain an affinity purification or attachment moiety; and

(ii) sequence determination or amplification reagents: the kit may also contain other suitably packaged reagents and materials needed for the particular sequencing amplification protocol. In one preferred embodiment, the kit comprises a panel of sequencing or amplification primers, whose sequences correspond to sequences adjacent to at least one of the polymorphic positions, as well as a means for detecting the presence of each polymorphic sequence.

In a particular embodiment, it is provided a kit which comprises a pair of nucleotide primers specific for amplifying all or part of the SMAD4 gene comprising at least one of mutations that are identified herein, especially at the codon constituted by positions 1498-1500.

Alternatively, the kit of the invention may comprise a labelled compound or agent capable of detecting the mutated polypeptide of the invention (e.g., an antibody or aptamers as described above which binds the polypeptide). For example, the kit may comprise (1) a first antibody (e.g., attached to a solid support) which binds to a polypeptide comprising a mutation of the invention; and, optionally, (2) a second, different antibody which binds to either the polypeptide or the first antibody and is conjugated to a detectable agent.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a bone mineral density related disease.

Therapeutic Methods of the Invention:

In a further object, the invention relates to use, methods and pharmaceutical compositions for treating or preventing Myhre Syndrome.

Accordingly, the present invention relates to an inhibitor of the SMAD4-mediated TGFβ/BMP signalling pathway for use in the treatment of Myhre Syndrome.

As used herein the term “SMAD4-mediated TGFβ/BMP signalling pathway” has its general meaning in the art and refers to the pathway described in Massague J. TGF-beta signal transduction. Annu Rev Biochem. 1998; 67:753-91.

In one embodiment, the inhibitor of the SMAD4-mediated TGFβ/BMP signalling pathway is able to i) inhibiting the expression or activity of TGFβ, ii) inhibiting the expression or activity of BMP proteins, iii) inhibiting the expression or activity of SMAD4, iv) inhibiting the expression or activity of SMAD2, v) inhibiting the expression or activity of SMAD3, vi) inhibiting the expression or activity of SMAD5, or vii) inhibiting the expression or activity of SMAD8.

In a particular embodiment the inhibitor of the SMAD4-mediated TGFβ/BMP signalling pathway is selected from the group consisting of inhibitors of TGFβ expression, inhibitors of BMP expression, inhibitors of SMAD4 expression, inhibitors of SMAD2 expression, inhibitors of SMAD3 expression, inhibitors of SMAD5 expression and inhibitors of SMAD8 expression.

Inhibitors of expression for use in the present invention may be based on anti-sense oligonucleotide constructs. Anti-sense oligonucleotides, including anti-sense RNA molecules and anti-sense DNA molecules, would act to directly block the translation of the targeted mRNA by binding thereto and thus preventing protein translation or increasing mRNA degradation, thus decreasing the level of the targeted protein (e.g. SMAD4), and thus activity, in a cell. For example, antisense oligonucleotides of at least about 15 bases and complementary to unique regions of the mRNA transcript sequence encoding the targeted protein (e.g. SMAD4) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. The targeted protein (e.g. SMAD4) gene expression can be reduced by contacting a subject or cell with a small double stranded RNA (dsRNA), or a vector or construct causing the production of a small double stranded RNA, such that the targeted protein (e.g. SMAD4) gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developped by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the targeted protein (e.g. SMAD4) mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

For example, TGF beta antisense oligonucleotides are described in U.S. Pat. No. 5,683,988; U.S. Pat. No. 5,772,995; U.S. Pat. No. 5,821,234; U.S. Pat. No. 5,869,462; and WO 94/25588.

For example, SMAD4 antisense oligonucleotide are described in U.S. Pat. No. 6,013,787.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing the targeted protein (e.g. SMAD4). Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters.

In a particular embodiment, the inhibitor of the SMAD4-mediated TGFβ/BMP signalling pathway is a TGFβ antagonist.

The term “TGFβ antagonist” and its cognates such as “inhibitor,” “neutralizing,” and “downregulating” refer to a compound which acts as an antagonist of the biological activity of TGFβ. A TGFβ antagonist may, for example, bind to and neutralize the activity of TGFβ; affect the stability or conversion of the precursor molecule to the active, mature form; interfere with the binding of TGFβ to one or more receptors; or it may interfere with intracellular signalling of a TGFβ receptor. The term “direct TGFβ antagonist” generally refers to any compound that directly downregulates the biological activity of TGFβ. A molecule “directly downregulates” the biological activity of TGFβ if it downregulates the activity by interacting with a TGFβ gene, a TGFβ transcript, a TGFβ ligand, or a TGFβ receptor.

TGFβ antagonists are well known in the art (see e.g. Dominique Bonafoux, Wen-Chung Lee Strategies for TGF-β modulation: a review of recent patents. Expert Opinion on Therapeutic Patents December 2009, Vol. 19, No. 12, Pages 1759-1769: 1759-1769.)

Examples of TGFβ antagonists that may be used include but are not limited to antibodies, polypeptides and small organic molecules.

In another particular embodiment, the TGFβ antagonist according to the invention is an antibody or a portion thereof. Said antibody may be directed against TGFβ or TGFβ receptor.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of TGFβ or TGFβ receptor. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant TGFβ proteins may be provided by surface expression on recombinant cell lines. TGFβ may be provided in the form of human cells that express TGFβ. Recombinant forms of TGFβ may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Antibodies showing TGFβ antagonistic activities are part of the common general knowledge. For example, monoclonal and polyclonal antibodies directed against one or more isoforms of TGFβ have beed described in U.S. Pat. No. 5,571,714; WO 97/13844; and WO 00/66631; WO 05/097832; WO 05/101149; WO 06/086469. Antibodies directed against TGFβ receptors have also bee described in Flavell et al., Nat. Rev. Immunol. 2:46-53 (2002; U.S. Pat. No. 5,693,607; U.S. Pat. No. 6,001,969; U.S. Pat. No. 6,008,011; U.S. Pat. No. 6,010,872; WO 92/00330; WO 93/09228; WO 95/10610; and WO 98/48024.

In another embodiment, the TGFβ antagonist is a small organic molecule that may be useful are also well known to those of skill in the art, including, but not limited to, those described in WO 02/062753; WO 02/062776; WO 02/062787; WO 02/062793; WO 02/062794; WO 02/066462; WO 02/094833; WO 03/087304; WO 03/097615; WO 03/097639; WO 04/010929; WO 04/021989; WO 04/022054; WO 04/024159; WO 04/026302; WO 04/026871; U.S. Pat. No. 6,184,226; WO 04/016606; WO 04/047818; WO 04/048381; WO 04/048382; WO 04/048930; WO 04/050659; WO 04/056352; WO 04/072033; WO 04/087056 WO 05/010049; WO 05/0032481; WO 05/0065691; WO 05/092894; WO 06/026305; WO 06/026306; and WO 06/052568.

In another embodiment, the TGFβ antagonists may be a dominant negative TGFβ.

In another embodiment, the TGFβ antagonists may be soluble TGFβ receptors.

Other TGFβ antagonists also include but are not limited to LAP (WO 91/08291); LAP-associated TGFβ (WO 94/09812); TGFβ-binding glycoproteins/proteoglycans such as fetuin (U.S. Pat. No. 5,821,227); decorin, betaglycan, fibromodulin, lumican, and endoglin (U.S. Pat. No. 5,583,103; U.S. Pat. No. 5,654,270; U.S. Pat. No. 5,705,609; U.S. Pat. No. 5,726,149; U.S. Pat. No. 5,824,655; U.S. Pat. No. 5,830,847; U.S. Pat. No. 6,015,693; WO 91/04748; WO 91/10727; WO 93/09800; and WO 94/10187); mannose-6-phosphate or mannose-1-phosphate (U.S. Pat. No. 5,520,926); prolactin (WO 97/40848); insulinlike growth factor 11 (WO 98/17304); extracts of plants, fungi, and bacteria (EU 813875; JP 8119984; and U.S. Pat. No. 5,693,610); and any mutants, fragments, or derivatives of the above-identified molecules that retain the ability to inhibit the biological activity of TGFβ.

In a particular embodiment, the inhibitor of the SMAD4-mediated TGFβ/BMP signalling pathway is a BMP antagonist.

Examples of BMP antagonists that may be used include but are not limited to antibodies, polypeptides and small organic molecules.

In a particular embodiment, the BMP antagonist according to the invention is an antibody or a portion thereof. Said antibody may be directed against a BMP protein or a BMP receptor.

The inhibitors of the SMAD4-mediated TGFβ/BMP signalling pathway may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising inhibitors of the SMAD4-mediated TGFβ/BMP signalling pathway as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitors of the SMAD4-mediated TGFβ/BMP signalling pathway can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic b as e s as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

Inhibitors of the SMAD4-mediated TGFβ/BMP signalling pathway may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the inhibitors of the SMAD4-mediated TGFβ/BMP signalling pathway formulated for parenteral administration, such as intravenous or intramuscular injection, other pharmaceutically acceptable forms include, e.g. tablets or other solids for oral administration; liposomal formulations; time release capsules; and any other form currently used.

The invention further relates to a method for treating or preventing Myhre syndrome which comprises the step of administering a subject in need thereof with a SMAD4 polynucleotide, i.e. a nucleic acid sequence that encodes a wild-type SMAD4, so that SMAD4 is expressed in vivo by the cells of the subject that have been transfected with said polynucleotide. Accordingly, said method leads to an overexpression of wild-type SMAD4 which compensates expression of defective mutated SMAD4. The administered polynucleotide does not contain a mutation selected in the group consisting of c.1498 A>G, c.1499 T>C, and c. 1500 A>G.

The invention also relates to a SMAD4 polynucleotide for use in the treatment of Myhre syndrome. Preferably said SMAD4 polynucleotide is administered in a therapeutically effective amount.

Preferably the SMAD4 polynucleotide sequence according to the invention is associated with elements that enable for regulation of its expression, such as a promoter sequence.

Such a nucleic acid may be in the form of a vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The SMAD4 polynucleotide may be introduced into a target cell by means of any procedure known for the delivery of nucleic acids to the nucleus of cells, ex vivo, on cells in culture or removed from an animal or a patient, or in vivo.

Ex vivo introduction may be performed by any standard method well known by one skilled in the art, e.g. transfection, electroporation, microinjection, transduction, cell fusion, DEAE dextran, calcium phosphate precipitation, or use of a gene gun.

The SMAD4 polynucleotide can also be introduced ex vivo or in vivo by lipofection. In certain embodiments, the use of liposomes and/or nanoparticles is contemplated for the introduction of the donor nucleic acid targeting system into host cells.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core.

Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes (Felgner et al., 1989).

Alternatively, one of the simplest and the safest way to deliver SMAD4 polynucleotide across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin O, cholera toxin, and recombinant modified labile toxin of E coli; and polysaccharides, such as glucose, sucrose, fructose, or maltose, for instance, which act by disrupting the osmotic pressure in the vicinity of the cell membrane. Other methods have been described to enhance delivery of free polynucleotides, such as blocking of polynucleotide inactivation via endo- or exonucleolytic cleavage by both extra- and intracellular nucleases.

Alternatively, the invention also provides a method for treating or preventing Myhre syndrome which comprises the step of administering a subject in need thereof with a wild-type SMAD4.

The administered SMAD4 polypeptide may be a variant of the wild-type SMAD4, provided that the polypeptide retains the same activity. Accordingly; the polypeptide does not comprise a mutation selected from the group consisting of mutations which result in I500M, I500T and I500V mutants of SMAD4.

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Polypeptides of the invention can be use in an isolated (e.g., purified) form or contained in a vector, such as a membrane or lipid vesicle (e.g. a liposome).

The invention will be further illustrated by the following figures and examples. However, these examples and figures should not be interpreted in any way as limiting the scope of the present invention.

### Example

### Material & Methods

Patients.

A total of 11 MS cases were included in the study. They all fulfilled the following inclusion criteria: short stature, short hands restricted joint limitation, thick skin, characteristic facial features, deafness and developmental delay (Table 1).

Exome Sequencing.

Massively parallel sequencing: DNA (3 μg) extracted from XYZ cells from the patient was sheared with a Covaris S2 Ultrasonicator (Covaris). An adapter-ligated library was prepared with the Paired-End Sample Prep kit V1 (Illumina). Exome capture was performed with the SureSelect Human All Exon kit (Agilent Technologies). Single-end sequencing was performed on an Illumina Genome Analyzer IIx (Illumina), generating 72-base reads. Sequence alignment, variant calling and annotation: The sequences were aligned to the human genome reference sequence (hg18 build), using BWA aligner (Li and Durbin, 2009). Downstream processing was carried out with the Genome analysis toolkit (GATK; McKenna et al., 2010), SAMtools (Li et al., 2009) and Picard Tools (http://picard.sourceforge.net). Substitution calls were made with a GATK UnifiedGenotyper, whereas indel calls were made with a GATK IndelGenotyperV2. All calls with a read coverage ≦2× and a Phred-scaled SNP quality of ≦20 were filtered out. All the variants were annotated using an in-house developed annotation software system.

Mutation Detection.

We designed a series of 12 intronic primers to amplify the 11 coding exons of SMAD4. We purified the amplicons and sequenced them using the fluorescent dideoxy-terminator method on an automatic sequencer (ABI 3100).

Three dimensional structure of the human SMAD4 was modelled by comparative modelling methods and energy minimization using the program Swiss model in the optimized mode (using Swiss-Pdb Viewer 3.7)

Western Blotting Analysis.

For western blot of pSmad2/3 and pSmad1/5/8, cell lysates were obtained from skin fibroblasts (control and GD/AD patients) and anti-actin (Invitrogen), anti-pSmad2/3 or anti pSmad1/5/8 (Cell signalling technology) antibodies were used. Respective protein species were quantified by densitometry (Kodak 1D image analysis software).

Results

Myhre syndrome (OMIM #139210, MS) is a developmental disorder characterized by short stature, short hands and feet, facial dysmorphism, muscular hypertrophy, deafness and cognitive delay. Using exome sequencing in MS cases, we selected mothers against DPP homolog 4 (SMAD4) as a candidate gene based on its pivotal role in BMP and TGFβ signalling. We identified 3 distinct heterozygous missense SMAD4 mutations at codon Isoleucine 500 in 11 MS cases. All 3 mutations were located in the Mad Homology 2 (MH2) domain. We found increased levels of SMAD4 and enhanced SMAD2/3 and SMAD1/5/8 phosphorylation in patient fibroblasts. These results provide evidence of dysregulation of BMP and TGFβ signalling as the underlying mechanism of MS phenotype.

MS is a well defined entity characterized by pre and postnatal short stature, brachydactyly, facial dysmorphism, (short palpebral fissures, maxillary hypoplasia, prognathism, short philtrum), thick skin, generalized muscle hypertrophy, and restricted joint mobility. Deafness of mixed conductive and sensory type is consistently observed in older patients. Other features include developmental delay with mental retardation or/and behavioral disturbance, cardiac defects, cryptorchidism and bone anomalies. Skeletal manifestations include thickened calvarium, cone-shaped epiphyses, shortened tubular bones, hypoplastic iliac wings, broad ribs and large and flattened vertebrae with large pedicles. Differential diagnoses include acromicric and geleophysic dysplasias (AD/GD), also characterized by short stature, brachydactyly, joint limitation and skin thickening. MS can be distinguished from AD and GD by facial features, the presence of developmental delay, deafness and distinct skeleton features. We have recently identified ADAMTSL2 mutations in GD and demonstrated a direct involvement of ADAMTSL2 in TGFβ bioavailability. The molecular basis of AD are still unknown.

All reported MS cases are sporadic and advanced paternal age at the time of fecondation has been reported in a few instances supporting de novo dominant mutations.

We collected DNA samples from 11 patients all fulfilling the diagnostic criteria for MS (Table 1). To identify the disease gene, we performed exome sequencing in 2/11 MS cases. We first focused our analyses on non synonymous variants, splice acceptor and donor site mutations and coding indels, anticipating that synonymous variants were far less likely to be pathogenic. We regarded variants as previously unidentified if they were absent from control populations and from all datasets, including dbSNP129, the 1000 Genomes Project, and “homemade” exome data.

Under a dominant model, exome analysis led to highlight 19 candidate genes. Considering our previous findings of enhanced TGFβ signaling in GD, we selected mothers against DPP homolog 4 (SMAD4) as the best candidate gene. Exome analysis detected the same heterozygous missense SMAD4 mutation in the two MS patients tested (c.1498A>G; p. I500T). These results were confirmed by Sanger sequencing.

The 11 coding exons of SMAD4 encode a 552 residue protein composed of a Mad Homology 1 (MH1) domain, a linker and a MH2 domain. Direct sequence analysis of the coding regions in 9 additional MS cases led to the identification of three missense mutations in the MH2 domain of SMAD4 all involving an Isoleucine residue at position 500 (p.I500T; p.I500V; p.I500M) (Table 2). They were considered as pathogenic in the PolyPhen database and were highly conserved across species. The base changes were absent from 200 ethnicity-matched controls and were not observed in MS parents, confirming that they occurred de novo.

Smad4 belongs to the eight member family of Smad proteins, divided into three functional classes: the receptor-regulated Smads (R-SMADs, Smad1, 2, 3, 5 and 8), the co-mediator Smad (Smad4) and the inhibitory Smads (Smad6 and Smad7). Smad2 and Smad3 respond to TGFβ and activin, while Smad1, 5 and 8 function in BMP signalling pathways. Receptor-regulated Smads form heterodimers with Smad4 and then translocate into the nucleus to induce or repress the expression of TGFβ target genes.

To determine the functional impact of SMAD4 mutations, we assessed the level of SMAD4 synthesis in cultured skin fibroblasts using protein blot analysis. We found an enhanced level of SMAD4 in MS patient fibroblasts, contrasting with a weak endogenous level of SMAD4 in age- and passage-matched control skin fibroblasts. This findings suggest that the isoleucine 500 variants markedly alter the stability of mutated SMAD4.

Considering the pivotal role of SMAD4 in TGFβ and BMP signalling, we analysed the level of phosphorylation of SMAD2/3 and SMAD1/5/8 in MS patient fibroblast lysates. We found an eightfold higher amount of phosphorylated SMAD2/3 (pSMAD2/3) and eleven fold higher amount of phosphorylated SMAD1/5/8 (pSMAD1/5/8) in patients compared to control cell lysates.

Here, we report the identification of heterozygous SMAD4 mutations in 11 patients presenting with the characteristic features of Myhre syndrome. Based on the observation of de novo mutations in all cases tested, we demonstrate the dominant mode of inheritance of MS. Moreover, based on the observation of heart, uro-genital, intestinal, velopharyngeal and eye anomalies in our patients, we expande the phenotypic spectrum of this disorder and give support to the key role of SMAD4 during development.

Interestingly, homozygous Smad4 deficient mice died before day7.5 but mutant embryos have reduced size (Sirad et al, 1998). Conditional knockout of Smad4 in chondrocytes (smad4^{co/co}) results in dwarfism with severely disorganised growth plate and decreased expression of Indian hedgehog/PTHrP signaling supporting the view that smad4-mediated TGFβ signalling is required to insure the normal organisation of chondrocytes in the growth plate (Zhang et al. 2005). The smad4^{co/co} are also characterized by a smaller cochlear volume. Anomalies of the osseous spiral lamina and basilar membrane lead to severe sensorineural hearing loss (Yang 2009) as observed in MS supporting the role of smad4 in innner ear development and normal auditory function. Finally, the targeted ablation of smad4 in osteoblasts revealed that smad4 is also required for maintaining normal postnatal bone homeostasis in mice. This function may explain the thick calvarium and facial features observed in MS patients (including prognatism and maxillary hypoplasia).

This is the first report of a unique amino acid change involving the Isoleucine 500 located in the MH2 domain in a developmental disorder. Up till now, SMAD4 has been considered as a tumor suppressor. SMAD4 loss of function mutations have been reported in juvenile polyposis syndrome characterized by the presence of juvenile polyps in the gastrointestinal tract and increased colorectal cancer risk (Jass et al. 1988). Inactivation of SMAD4 has also been demonstrated in pancreatic and colorectal carcinoma (Schutte et al. 1996 and Hahn et al. 1996). Tumors lacking functional SMAD4 tend to be more invasive, angiogenic and consequently are more likely to form metastatic lesions (Sunamura et al. 2002). It is admitted that disruption of TGFβ signaling contributes to the formation of human malignancies (Kurokawa et al. 1998).

By contrast, our findings of increased SMAD4 levels of and enhanced SMAD2/3 and SMAD1/5/8 phosphorylation in patient fibroblasts support activation of TGFβ and BMP signalling in Myhre syndrome. These findings are in agreement with our previous observation of an enhanced TGFβ signalling in geleophysic dysplasia, a closely related condition also characterized by short stature, short hands, stiff skin and restricted joint limitation and further support the specific bone and cartilage consequences of enhanced TGFβ signalling in this group of disorders.

Ongoing studies will hopefully contribute to further elucidate the context-dependent mechanisms and tissue-specific consequences of smad4-mediated TGFβ/BMP signalling and inspire novel pharmacological strategies aimed at targeting those complex pathways.

TABLE 1
Clinical and radiological features of the 11 patients with Myhre syndrome

| | 1 | 2 | 3 | 4 | 5 | 6 |
|---|---|---|---|---|---|---|
| Origin | France | France | France | Belgium | Belgium | Netherlands |
| Sex | F | F | F | F | F | F |
| Age (years) | 10 | 12 | 12 | 11 | 16 | 22 |
| Height | −3 SD | −2SD | −2SD | −4 SD | −5 SD | −4.8 SD |
| Muscular hypertrophy | + | + | + | + | + | + |
| Joint limitation | + | + | + | + | + | + |
| Short hands/feet | + | + | + | +, 5th CD | + | + |
| Thick skin | + | + | − | + | + | + |
| Proguathism | + | + | + | + | + | + |
| Short philtrum | + | + | + | + | + | + |
| short palpebral fissures | + | + | + | + | + | + |
| Abnormal ears | small | + | − | + | + | small |
| Deafness | + | + | − | + | + | + |
| | Hearing aid | Hearing aid | (not tested) | Hearing aid | Hearing aid | Hearing aid |
| Developmental delay | + | + | mild | + | − | + (mild) |
| | | | | Hyperactivity | | Psychotic profile |
| | | | | Behavioral | | |
| | | | | troubles | | |
| Thick calvaria | + | + | Not done | + | + | + |
| Short tubular bones | + | + | | + | + | + |
| Flat vertebrae, | − | − | | − | + | − |
| Large pedicules | + | + | | + | + | + |
| Conc-shaped epiphyses | + | − | | + | − | − |
| Bone age | Delayed | Delayed | | Delayed | Normal | Delayed |
| Heart | PDA* | − | PDA | PDA, HTA | − | PDA, pulmonary |
| | | | | | | stenosis |
| Puberty | Premature | Premature | — | Premature | | Premature |
| | menarche | menarche | | menarche | | menarche |
| Other | Duodenal | Myopia | Vesico-ureteral | | | Velopharyngeal |
| | stenosis | retinopathy | reflux. | | | insufficiency |
| | Strabismus | | Srabismus | | | cheilognathoschisis |
| | Pulmonary | | | | | (left) |
| | infections | | | | | |

| | 7 | 8 | 9 | 10 | 11 |
|---|---|---|---|---|---|
| Origin | Switzerland | UK | France | France | Italy |
| Sex | F | M | M | F | M |
| Age (years) | 41/2 | 17 | 15 | 9 | 7 |
| Height | −3 SD | <−2 SD | −1.5 DS | −2 SD | −2 SD |
| Muscular hypertrophy | + | + | + | + | + |
| Joint limitation | ++ | + | + | + | + |
| Short hands/feet | + | + | + | + | + |
| Thick skin | ++ | − | + | + | + |
| Proguathism | + | + | + | + | + |
| Short philtrum | Mild | + | + | + | + |
| short palpebral fissures | − | + | + | + | + |
| Abnormal ears | + | − | − | − | − |
| Deafness | − | + | + | + hearing aid | − |
| | (no tested) | Ossicular | | Conduction | |
| | | fixation | | Ossicular | |
| | | | | dysplasia | |
| Developmental delay | + | + | + (mild) | mild | + |
| | | | Hyperactivity | | |
| | | | Psychotic | | |
| | | | profile | | |
| Thick calvaria | + | + | + | + | Not done |
| Short tubular bones | + | + | + | + | |
| Flat vertebrae, | + | + | + | − | |
| Large pedicules | + | + | + | + | |
| Conc-shaped epiphyses | + | + | − | + | |
| Bone age | − | ? | Normal | delayed | |
| Heart | Ventricular non | PDA, pulmonary | | HTA | Recurrent |
| | compaction | stenosis, HTA | | | pericarditis |
| Puberty | — | — | | Premature | — |
| | | | | menarche | |
| Other | Strabismus, | — | Strabismus | Velopharyngeal | Pulmonary |
| | astigmatism | | Astigmatism | insufficiency | infections |
| | | | Sacral dimple | | |

* [symbol] [symbol]
indicates data missing or illegible when filed

TABLE 2
SMAD4 mutations identified in cases with Myhre Syndrome

| | Nucleotide | Aminio Acid |
|---|---|---|
| Family | change | change |
| 1 | c.1499 T > C | p.I500T |
| 2 | c.1499 T > C | p.I500T |
| 3 | c.1498 A > G | p.I500V |
| 4 | c.1499 T > C | p.I500T |
| 5 | c.1498 A > G | p.I500V |
| 6 | c.1498 A > G | p.I500V |
| 7 | c.1499 T > C | p.I500T |
| 8 | c.1500 A > G | p.I500M |
| 9 | c.1498 A > G | p.I500V |
| 10 | c.1498 A > G | p.I500V |
| 11 | c.1499 T > C | p.I500T |

### REFERENCES

Throughout this application, various references describe the state of the art to which this invention pertains. The disclosures of these references are hereby incorporated by reference into the present disclosure.
Allah S. et al. Molecular screening of ADAMTSL2 gene in 33 patients reveals thegenetic heterogeneity of geleophysic dysplasia. J Med Genet. In press (2011)
Burglen L. et al. Myhre syndrome: new reports, review, and differential diagnosis. J. Med. Genet. 40, 546-551 (2003)
Hahn SA DPC4, a candidate tumor suppressor gene at human chromosome 18q21.1. Science. 271, 350-353 (1996)
Howe JR. et al. Mutations in the SMAD4/DPC4 gene in juvenile polyposis. Science.; 280, 1086-1088 (1998)
Kurokawa M. et al. The oncoprotein Evi-1 repress TGFbeta signaling by inhibitingSmad3. Nature. 394, 92-96 (1998)
Le Goff C. et al. ADAMTSL2 mutations in geleophysic dysplasia demonstrate a rolefor ADAMTS-like proteins in TGF-β bioavailability regulation. Nat. Genet. 40, 1119-1123 (2008)
Myhre SA., Ruvalcaba HA., Graham CB. A new growth deficiency syndrome. Clin. Genet. 20, 1-5 (1981)
Ross S. and Hill C. S. How the Smads regulate transcription. Int. J. of Biochem. & CellBiol. 40, 383-408 (2008)
Schutte M. et al. DPC4 gene in various tumor types. Cancer Res. 56, 2527-2530 (1996)
Shi Y. and Massague J. Mechanisms of TGF-beta signaling from cell membrane to thenucleus. Cell. 113, 685-700 (2003)
Sirard C. et al. The tumor suppressor gene Smad4/Dpc4 is required for gastrulation and later anterior development of the mouse embryo. Genes Dev. 12, 107-119 (1998)
Sunamura M. et al. Gene therapy for pancreatic cancer based on geneticcharacterization of the disease. J. Hepatobiliary Pancreat Surg. 9, 32-38 (2002)
Tan X. et al. Smad4 is required for maintaining normal murine postnatal bonehomeostasis. J. of Cell Science. 120, 2162-2170 (2007)
Yang SM. et al. Chondrocyte-specific Smad4 gene conditional knockout results inhearing loss and inner ear malformation in mice. Dev. Dyn. 238, 1897-1908 (2009)
Zhang J. et al. Smad4 is required for the normal organization of the cartilage growthplate. Dev. Biol. 284, 311-322 (2005)

1-8. (canceled)

9. The method of claim 13, wherein said inhibitor of the SMAD4-mediated TGFβ/BMP signalling pathway is selected from the group consisting of inhibitors of TGFβ expression, inhibitors of BMP expression, inhibitors of SMAD4 expression, inhibitors of SMAD2 expression, inhibitors of SMAD3 expression, inhibitors of SMAD5 expression and inhibitors of SMAD8 expression.

10. The method of claim 13, wherein said inhibitor of the SMAD4-mediated TGFβ/BMP signalling pathway is a TGFβ antagonist.

11. A method for diagnosing or predicting Myhre Syndrome, or a risk of Myhre Syndrome, in a subject, which method comprises
detecting, in a sample from the subject, a mutation in a SMAD4 gene by:
i) isolating nucleic acid encoding the SMAD4 gene from the sample;
ii) contacting isolated nucleic acid with oligonucleotide primers specific for amplifying, in the SMAD4 gene: A at position 1498, G at position 1498, T at position 1499, C at position 1499, A at position 1500 and G at position 1500, and
if amplification products generated in said step of contacting indicate that A is present at position 1498, T is present at position 1499 and A is present at position 1500 of the SMAD4 gene, then concluding that the subject does not have and is not at risk of having Myhre Syndrome; and
if G is present at position 1498, or if C is present at position 1499 or if G is present at position 1500 of the SMAD4 gene, then concluding that said subject has or is at risk of having Myhre Syndrome.

12. A method for diagnosing or predicting Myhre Syndrome, or a risk of Myhre Syndrome, in a subject, which method comprises
detecting, in a sample from the subject, a mutation in SMAD4 gene by contacting SMAD4 protein in the sample with
i) antibodies that specifically recognize isoleucine (I) at position 500 in the SMAD4 protein (SEQ ID NO: 2),
ii) antibodies that specifically recognize methionine (M) at position 500 in the SMAD4 protein (SEQ ID NO: 2),
iii) antibodies that specifically recognize threonine (T) at position 500 in the SMAD4 protein (SEQ ID NO: 2), and
iv) antibodies that specifically recognize valine (V) at position 500 in the SMAD4 protein (SEQ ID NO: 2); and,
if M, T or V is detected at position 500 in the SMAD4 protein (SEQ ID NO: 2), then concluding that said subject has or is at risk of having Myhre Syndrome; and
if I is detected at position 500 in the SMAD4 protein (SEQ ID NO: 2), then concluding that said subject does not have and is not at risk of having Myhre Syndrome.

13. A method of treating Myhre Syndrome in a subject in need thereof, comprising
administering to said subject a therapeutically effective amount of an inhibitor of the SMAD4-mediated transforming growth factor β/bone morphogenic protein (TGFβ/BMP) signaling pathway.


SEQUENCE LISTING (2 sequences)

SEQ ID NO: 1 - DNA, 1659 bp, Homo sapiens
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactga

SEQ ID NO: 2 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD